(19) United States
(12) Patent Application Publication
(10) Pub. No.: US 2025/0019339 A1
(43) Pub. Date: Jan. 16, 2025

(54) SOLVENT COMPOUNDS FOR USE AS REPLACEMENTS FOR SLOW EVAPORATING SOLVENTS

(71) Applicant: TBF ENVIRONMENTAL TECHNOLOGY INC., Delta (CA)

(72) Inventors: David A. PASIN, Vancouver (CA); Joseph Mitchell CLARKSON, Vancouver (CA); Matthew D. TEWKESBURY, Vancouver (CA); Laurel L. SCHAFER, Vancouver (CA)

(21) Appl. No.: 18/882,145

(22) Filed: Sep. 11, 2024

Related U.S. Application Data
(63) Continuation of application No. 17/269,961, filed on Feb. 19, 2021.
(60) Provisional application No. 62/721,257, filed on Aug. 22, 2018.

(51) Int. Cl. C07C 69/96; C07C 68/06; C09D 7/20; C09D 9/00; C10M 105/48; C11D 7/26; C11D 7/50; C23G 5/032

(52) U.S. Cl. CPC C07C 69/96; C07C 68/06; C09D 7/20; C09D 9/005; C10M 105/48; C11D 7/266; C11D 7/5022; C23G 5/032; C10M 2207/325; C11D 2111/20

(57) ABSTRACT

The present disclosure provides, in part, a solvent compound for use as a slow evaporating replacement. More specifically, the present disclosure relates to volatile organic components (VOC)-exempt solvent compounds that may be used as a slow evaporating replacement that may, for example, be useful as a component or diluent in the production of paints and coatings, and other industrial products.

This application is a continuation of U.S. patent application Ser. No. 17/269,961, which is a national phase entry of International Patent Application No. PCT/IB2019/057015 filed on Aug. 20, 2019, which claims priority to U.S. Provisional Patent Application No. 62/721,257 filed on Aug. 22, 2018, which are hereby incorporated by reference.

### FIELD OF THE INVENTION

The present disclosure relates generally to solvent compounds. More specifically, the present disclosure relates to VOC-exempt solvent compounds that may be used to replace slow evaporating solvents.

### BACKGROUND OF THE INVENTION

Smog is known to have negative health effects on humans and the environment. A major contributor to smog formation is the release of volatile organic compounds (VOCs) which are emitted from many sources including automobile exhaust and organic solvents. VOCs are defined as “any compound of carbon, excluding carbon monoxide, carbon dioxide, carbonic acid, metallic carbides or carbonates, and ammonium carbonate, which participates in atmospheric photochemical reactions”. Numerous consumer products contain VOCs as an integral component of the consumer product's function or application, such as paints or chemical coating strippers. To combat the adverse effects VOCs have on air quality in North America, agencies such as Environment and Climate Change (Canada) and the Environmental Protection Agency (United States) enforce limits on VOC content in manufacturing workplaces and consumer products. VOC emission limits in some municipalities have become even more stringent than federal standards. For example, the South Coast Air Quality Management District (SCAQMD), which regulates VOC emissions in and around Orange County, California, has found success in reducing smog levels by half since the 1980's despite population growth in the area. Such successes inspire increased awareness and provide support for SCAQMD's mission. While increased awareness and enforcing limits on VOC content has helped combat smog formation significantly, many sources of VOC emissions have not been curtailed. Replacing solvents that are known to contribute heavily to smog formation, due to high VOC content, with solvents that have zero or low VOC content are thus highly sought after. To further the health and safety of their constituents some agencies have also reviewed the toxicity of commonly used chemicals. In Canada, the use of solvents and paints alone corresponds to 15% of all VOC emissions, with 314.0 kilotonnes in 2014, making it the second largest contributor next to the oil and gas industry (734.1 kilotonnes in 2014). Since the VOC's used in paints and coatings are released into the environment, they should be as biodegradable and non-toxic as possible. Although some zero or low VOC solvents exist in the marketplace, their cost and limited applicability reduce their wide-spread use.

### SUMMARY OF THE INVENTION

In one aspect, the present invention provides a compound of Formula (I):

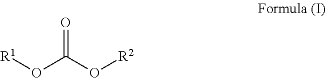

where R¹ and R² may each independently be: C₁₋₁₂ alkyl, C₂₋₁₂ alkenyl, C₂₋₁₂ alkynyl, C₆₋₁₄ aryl or siloxy, each of which may be independently optionally substituted, for use as a replacement for a slow evaporating solvent.

In some embodiments, the C₁₋₁₂ alkyl, C₂₋₁₂ alkenyl, C₂₋₁₂ alkynyl or C₆₋₁₄ aryl are substituted with a heteroatom, such as N, O or S.

In some embodiments, R¹ or R² may each independently be hexyl, heptyl, octyl, or decyl.

In some embodiments, R¹ may be methyl and R² may be hexyl, heptyl, octyl, or decyl.

In some embodiments, R¹ may be methyl and R² may be 2-ethylhexyl.

In some embodiments, the compound may be:

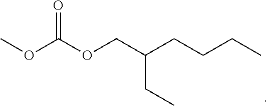

In some embodiments, the slow evaporating solvent may be an aliphatic solvent, an aromatic solvent, a glycol, or a ketone type solvent. In some embodiments, the slow evaporating solvent may be Mineral (white) Spirits, Aromatic 100, Aromatic 150, or Methyl Amyl Ketone.

In some embodiments, the compound may be useful as a component or diluent in the production of a paint or a coating; as a component in the formulation of general industrial primer, intermediate and/or topcoat; in the manufacture of a roof repair product; in the manufacture of a foundation repair product; as a metal cleaner or a component thereof; as a metal degreaser or a component thereof; as a paint stripper or a component thereof; as a diluent; as a thickener; or as a solvent.

The paint or coating may be an architectural paint or coating, a marine paint or coating, an aerospace paint or coating, an industrial paint or coating, a commercial paint or coating, a concrete paint or coating and/or a residential paint or coating.

The solvent may be a retarding solvent, an extraction solvent, a degreasing solvent, a cleaning solvent, or a component thereof.

The cleaning solvent may be an industrial cleaning solvent, a commercial cleaning solvent, or a residential cleaning solvent.

The solvent may be useful for dissolving tar or asphalt, for removing marks on flooring, or for removing carbon, grease, grime, gum, paint, adhesive or adhesive residue.

The diluent or solvent may be useful in liquid, aerosol paint, marking or adhesive formulations.

In some embodiments, the compound may be a sealant, a penetrant, a lubricant, a colourant diluent or a colourant dispersant.

In some embodiments, the compound may be useful as a substitute for an ester alcohol, or as a reactive intermediate in the formation of an ester derivative.

In some embodiments, the compound may be useful as an inert ingredient in the formulation of an insecticide, a fungicide and/or a rodenticide.

In some aspects, the present invention provides a kit or commercial package including a compound as described herein, together with instructions for use.

In some aspects, the present invention provides a method of forming a coating on a substrate, by incorporating a compound of Formula (I):

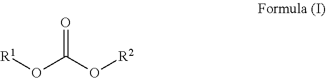

where R¹ and R² may each independently be: C₁₋₁₂ alkyl, C₂₋₁₂ alkenyl, C₂₋₁₂ alkynyl, C₆₋₁₄ aryl or siloxy, each of which may be independently optionally substituted, for use as a replacement for a slow evaporating solvent.

In some embodiments, the compound of Formula (I) may be provided in admixture with a paint.

In some aspects, the present invention provides a method preparing a compound as described herein as set forth in Example 2.

Other aspects and features of the present disclosure will become apparent to those ordinarily skilled in the art upon review of the following description of specific examples.

### DETAILED DESCRIPTION

The present disclosure provides, in part, compounds useful as a replacement for a slow evaporating solvent.

In some embodiments, the present disclosure provides a compound of Formula (I):

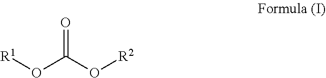

where R¹ and R² may each independently be: C₁₋₁₂ alkyl, C₂₋₁₂ alkenyl, C₂₋₁₂ alkynyl, C₆₋₁₄ aryl or siloxy, each of which may be independently optionally substituted, for use as a replacement for a slow evaporating solvent.

The compound may be:

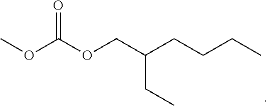

“Alkyl” refers to a straight or branched hydrocarbon chain group consisting solely of carbon and hydrogen atoms, containing no unsaturation and including, for example, from one to twelve carbon atoms, such as 1, 2, 3, 4, 5, 6, 7, 8, 9, 10, 11 or 12 carbon atoms, and which is attached to the rest of the molecule by a single bond. The alkyl chain can be branched or unbranched. Unless stated otherwise specifically herein, the alkyl group may be optionally substituted by one or more substituents as described herein. Unless stated otherwise specifically herein, it is understood that the substitution can occur on any carbon of the alkyl group.

“Alkenyl” refers to a straight or branched hydrocarbon chain group consisting solely of carbon and hydrogen atoms, containing at least one double bond and including, for example, from two to twelve carbon atoms, such as 2, 3, 4, 5, 6, 7, 8, 9, 10, 11 or 12 carbon atoms, and which is attached to the rest of the molecule by a single bond. The alkenyl chain can be branched or unbranched. Unless stated otherwise specifically herein, the alkenyl group may be optionally substituted by one or more substituents as described herein. Unless stated otherwise specifically herein, it is understood that the substitution can occur on any carbon of the alkenyl group.

“Alkynyl” refers to a straight or branched hydrocarbon chain group consisting solely of carbon and hydrogen atoms, containing at least one triple bond and including, for example, from two to twelve carbon atoms, such as 2, 3, 4, 5, 6, 7, 8, 9, 10, 11 or 12 carbon atoms, and which is attached to the rest of the molecule by a single bond. The alkynyl chain can be branched or unbranched. Unless stated otherwise specifically herein, the alkynyl group may be optionally substituted by one or more substituents as described herein. Unless stated otherwise specifically herein, it is understood that the substitution can occur on any carbon of the alkynyl group.

“Aryl” refers to a mono- or bicyclic aromatic ring containing only carbon atoms, including for example, 6-14 members, such as 6, 7, 8, 9, 10, 11, 12, 13, or 14 members. Examples of aryl groups include phenyl, biphenyl, naphthyl, indanyl, indenyl, tetrahydronaphthyl, 2,3-dihydrobenzofuranyl, dihydrobenzopyranyl, 1,4-benzodioxanyl, and the like. Unless stated otherwise specifically herein, the term “aryl” is meant to include aryl groups optionally substituted by one or more substituents as described herein. Unless stated otherwise specifically herein, it is understood that the substitution can occur on any carbon of the aryl group.

“Optional” or “optionally” means that the subsequently described event of circumstances may or may not occur, and that the description includes instances where the event or circumstance occurs one or more times and instances in which it does not. For example, “optionally substituted alkyl” means that the alkyl group may or may not be substituted and that the description includes both substituted alkyl groups and alkyl groups having no substitution, and that the alkyl groups may be substituted one or more times. Examples of optionally substituted alkyl, alkenyl or alkynyl groups include, without limitation, C₁₋₁₂ alkyl, C₂₋₁₂ alkenyl, C₂₋₁₂ alkynyl, C₆₋₁₄ aryl or siloxy groups. Examples of optionally substituted aryl groups include, without limitation, C₁₋₁₂ alkyl, C₂₋₁₂ alkenyl, C₂₋₁₂ alkynyl, C₆₋₁₄ aryl or siloxy groups, where the C₁₋₁₂ alkyl, C₂₋₁₂ alkenyl, C₂₋₁₂ alkynyl groups may include heteroatoms, such as N, O, or S, in the carbon chain.

In some embodiments, a compound according to the present disclosure may have a slow evaporation rate, for example, an evaporation rate of about 0.04. In some embodiments, a compound according to the present disclosure may have an evaporation rate between about 0.01 to about 0.08 or any value therebetween.

In some embodiments, a compound according to the present disclosure may have a high boiling point, for example, a boiling point over about 216° C. In some embodiments, a compound according to the present disclosure may have a boiling point between about 200° C. to about 290° C., or any value therebetween.

In some embodiments, a compound according to the present disclosure may have a low vapour pressure, for example, a vapour pressure below 0.04 Pa. In some embodiments, a compound according to the present disclosure may have a vapour pressure between about 0.02 Pa to about 0.09 Pa, or any value therebetween.

In some embodiments, a compound according to the present disclosure may have a flash point, for example, a flash point over 85° C. In some embodiments, a compound according to the present disclosure may have a flash point between about 65° C. to about 150° C., or any value therebetween.

In some embodiments, a compound according to the present disclosure may have a specific gravity, for example, a specific gravity over 0.916 In some embodiments, a compound according to the present disclosure may have a specific gravity between about 0.850 to about 1.10 or any value therebetween.

In some embodiments, a compound according to the present disclosure may have a low freezing point, for example, a freezing point below −50° C.

In some embodiments, a compound according to the present disclosure may be hydrolytically stable, for example, as observed by placing the compounds in water and confirming their structure by ¹H-NMR spectroscopy. By “hydrolytically stable” is meant that the compound does not exhibit substantial decomposition i.e., less than about 5% decomposition when placed in water. In some embodiments, a compound according to the present disclosure may exhibit about 0% to about 5% decomposition, or any value therebetween, when placed in water.

In some embodiments, a compound according to the present disclosure may have high hydrophobicity, for example, does not readily dissolve in water. Hydrophobicity may be measured using standard techniques, for example, by determining the solubility constant of the compound in water. By “high hydrophobicity” is meant a solubility constant of 99% or more. In some embodiments, a compound according to the present disclosure may have a hydrophobicity (i.e., solubility constant) between about 0% to about 99.9%, or any value therebetween.

In some embodiments, a compound according to the present disclosure may not be classified as hazardous air pollutants (HAPs), or as containing Saturates, Asphaltenes, Resins and Aromatics (SARA). In some embodiments, a compound according to the present disclosure may be VOC-exempt. In some embodiments, a compound according to the present disclosure may be a zero VOC or a VOC-compliant compound.

By “about” is meant a variance (plus or minus) from a value or range of 5% or less, for example, 0.5%, 1%, 1.5%, 2.0%, 2.5%, 3.0%, 3.5%, 4.0%, 4.5%, 5.0%, etc.

In some embodiments, a compound according to the present disclosure may have low toxicity as determined, for example by one or more of oral LD₅₀ on rats, biodegradability, teratogenicity, carcinogenicity and/or hepatic and renal toxicity measurements, which can be determined using standard methods. In some embodiments, a compound according to the present disclosure may contain reagents classified as non-carcinogenic. A compound according to the present disclosure may have an LD₅₀ of 5000 mg/kg or more.

In some embodiments, a compound according to the present disclosure may be substantially anhydrous, for example, containing less than 0.05 wt % water. In alternative embodiments, a compound according to the present disclosure may contain less than 500 ppm of water.

In some embodiments, a compound according to the present disclosure may have a purity of, for example, at least 99.5%, for example, at least 99.6%, 99.7%, 99.8%, 99.9%, or 100%.

In some embodiments, a compound according to the present disclosure may be useful as a replacement for slow evaporation solvents such as aliphatic solvents, aromatic solvents, glycols, ketone type solvents. In some embodiments, a compound according to the present disclosure may be useful as a replacement for slow evaporation solvents such as Mineral (white) Spirits, Aromatic 100, Aromatic 150, Methyl Amyl Ketone, etc.

In some embodiments, a compound according to the present disclosure may be useful as a component or diluent in the production of paints and coatings, such as architectural, marine, aerospace, industrial, commercial, concrete and/or residential coatings, for example: wall, trim, primer, roof, foundation, deck or stain paints and coatings.

In some embodiments, a compound according to the present disclosure may be useful in the manufacture of roof or foundation repair products.

In some embodiments, a compound according to the present disclosure may be useful as a solvent, such as an extraction solvent, a degreasing solvent or a cleaning solvent, for example an industrial, commercial, or residential cleaning solvent. In some embodiments, a compound according to the present disclosure may be useful in cleaning and/or degreasing applications.

In some embodiments, a compound according to the present disclosure may be useful as a solvent for dissolving tar or asphalt.

In some embodiments, a compound according to the present disclosure may be useful as a component of metal cleaners or a degreasers or as a metal cleaner and/or degreaser neat.

In some embodiments, a compound according to the present disclosure may be useful in the removal of marks on flooring, such as skid marks.

In some embodiments, a compound according to the present disclosure may be useful in the removal of carbon, grease, grime, gum, paint, adhesive or adhesive residue.

In some embodiments, a compound according to the present disclosure may be useful as a component of paint stripper or neat.

In some embodiments, a compound according to the present disclosure may be useful as a diluent and/or solvent in liquid or aerosol paint, marking and adhesive formulations.

In some embodiments, a compound according to the present disclosure may be useful as a sealant, a penetrant, colourant diluent or a colourant dispersant.

In some embodiments, a compound according to the present disclosure may be useful in synthetic/petrochemical grease, lubricant and/or penetrant applications.

In some embodiments, a compound according to the present disclosure may be useful in the manufacture and/or repair of asphaltic road materials (for example, blacktop or blacktop patch/repair).

In some embodiments, a compound according to the present disclosure may have utility as a component or diluent in the product and or in the manufacturing process for the manufacture of shoe polish, waterproofing compounds, sealants and glazes, tool/die cutting fluids, starter fluids, resins, lacquers, commercial/industrial/artist paints, coatings, adhesives, alkyd/urethane, asphalt, bitumen, silicone, metal work or textiles.

In some embodiments, a compound according to the present disclosure may be useful as a component of, or neat as, retarding solvent in, for example, coil coatings and high-bake enamel, oil field, floor polish, and/or wood preservatives formulations. By “retarding solvent” is meant a solvent capable of slowing down the drying time of a film to, for example, enhance film appearance and coverage.

In some embodiments, a compound according to the present disclosure may be useful as a substitute for an ester alcohol when used, for example, enhance thickening efficiency and/or act as a retarding solvent for use in coil coatings and/or high-bake enamels.

In some embodiments, a compound according to the present disclosure may be useful as a reactive intermediate in the formation of ester derivatives for a plasticizer.

In some embodiments, a compound according to the present disclosure may be useful to: create a film of high integrity; improve the overall performance characteristics of a paint or coating; enhance colour development of a film; improve gloss of a film; improve washability of a film; improve scrub resistance of a film; resist mud cracking of a film; and/or provide superior adhesion properties of a film.

In some embodiments, a compound according to the present disclosure may enhance the thickening efficiency of various organic and inorganic associative thickeners and rheology modifiers such as clays, Bentonites and fumed silicas, thereby improving the practical viscosity of a paint or coating.

In some embodiments, a compound according to the present disclosure may be widely useful as a component in the formulation of general industrial primer, intermediate and/or topcoat, as automotive refinish and/or OEM products, wood primer and/or topcoats, marine, can and/or coil, printing ink (for example, lithographic and/or letterpress) and/or oil field chemical (such as drilling mud, frothing agent, ore flotation) formulae.

In some embodiments, a compound according to the present disclosure may be used as an inert ingredient, which is permitted for non-food use contact, in the formulation of an insecticides, a fungicide and/or a rodenticide.

A compound according to the present disclosure may be prepared as described herein, or using techniques based on, or similar to, those known in the art, such as referenced in “Paint Formulations” by Ernest W. Flick (1988 Noyes, Publications).

### Example 1

### Synthesis of 2-ethylhexyl methyl carbonate (BibaSol)

The alcohol 2-ethylhexan-1-ol (450 mL, 2.88 mol) was added to a 2 L round bottom flask. The flask was then charged with hexanes (˜350 mL) and iron trichloride (2.5 g, 0.0154 mol). The reaction vessel was then agitated until the iron trichloride has been mostly dissolved. Dimethyl carbonate (740 mL, 8.78 mol) was then added. Boiling stones (3-10) are added to prevent bumping during the reaction. A Dean Stark apparatus was attached to the round bottom flask, and 15 mL of distilled water was added to the trap, the rest of the trap volume is filled with hexanes. A condenser was attached to the top of the Dean Stark apparatus. The reaction was then heated gently until the distillate temperature is 59 (+2° C.) As the distillate condenses into the Dean Stark trap the methanol formed from the transesterification reaction separates to the bottom of the trap. The trap was refreshed when the bottom layer occupies half of the Dean stark trap volume. The reaction was monitored by GC/MS and was continued until the 2-ethylhexan-1-ol is completely (or nearly) consumed. The reaction was filtered through basic alumina. The hexanes and dimethyl carbonate were then distilled off. The crude material was then distilled under vacuum (0.1-1.0 torr) and when the distillate reaches between 70-95° C., 2-ethylhexyl methyl carbonate was collected and analyzed for purity.

The physical properties of 2-ethylhexyl methyl carbonate (BibaSol) were determined to be as follows:
Specific Gravity: 0.916 (7.64 lbs/USG)
Evaporation rate: 0.04
Vapour pressure: 0.09 mm Hg @25° C.
Boiling Point: 216° C. (420.8° F.)
Flash Point: 85.5° C. (185.9° F.) PMCC—(Penske Martin Closed Cup)
Kauri-Butanol Value 77.5
Sustained combustion tests indicated that 2-ethylhexyl methyl carbonate (BibaSol) did not sustain combustion.

### Example 2

### Alternate Method of Synthesis of 2-Ethylhexyl Methyl Carbonate (BibaSol)

The alcohol 2-ethylhexan-1-ol (780 mL, 4.99 mol) was added to a 2 L round bottom flask. The flask was then charged with potassium tert-butoxide (5.5 g, 0.0490 mol). Dimethyl carbonate (1050 mL, 12.5 mol) was then added. Boiling stones (3-10) are added to prevent bumping during the reaction. A single piece distillation arm was attached to the round bottom flask, and the reaction was then heated gently until the distillate temperature is 65 (±2° C.) The reaction was monitored by GC/MS and was continued until the 2-ethylhexan-1-ol is completely (or nearly) consumed. The reaction was filtered through basic alumina to remove the potassium tert-butoxide catalyst. The remaining dimethyl carbonate was then distilled off. The crude material was then distilled under vacuum (0.1-1.0 torr) and when the distillate reaches between 70-95° C., 2-ethylhexyl methyl carbonate was collected and analyzed for purity.

### Example 3

### Solubilization Tests

2-ethylhexyl methyl carbonate (BibaSol) was tested against various materials. Table 1 shows materials that were solubilized by 2-ethylhexyl methyl carbonate.

TABLE 1

| Solvated | | | |
|---|---|---|---|
| Known | | | |
| Material | Type of Resyn | Solvents typically used | |
| Chlorinated | Pale yellow resin | chlorinated | Industrial corrosion |
| rubber | prepared by | solvents/Ketones/Xylene/Toluene/PCBTF | resistant |
| | adding up to 65% | | coatings/Pool |
| | chlorine to an | | Coatings |
| | elastomer | | |
| Hypalon | Synthetic rubber | chlorinated | Roofing material, |
| | made of | solvents/Ketones/Xylene/Toluene/PCBTF | inflatible boats |
| | chlorinated and | | |
| | sulfonated | | |
| | polyethylene | | |
| Kraton | Styrene-ethylene- | chlorinated | Thickening agent |
| 1726X | butylene-styrene | solvents/Ketones/Xylene/Toluene/PCBTF | used in afehsives, |
| | (SEBS) linear | | coatings and foam |
| | polymer (tri-block | | |
| | structure) | | |
| Sukorez | Hydrogenated | chlorinated | Adhesive tackifier |
| 100 | dicyclopentadiene | solvents/Ketones/Xylene/Toluene/PCBTF | (used in peel off |
| | (c9) hydrocarbon | | adhesives) |
| | resin. | | |
| | (Thermoplastic) | | |
| Globalprene | Styrene Isoprene | chlorinated | Hot melt & solvent |
| 5516 | Styrene Block | solvents/Ketones/Xylene/Toluene/PCBTF | based adhesives |
| | Copolymer (SIS) | | |
| Globalprene | Styrene Ethylene | chlorinated | Used in adhesives, |
| 9551 | Butylene Styrene | solvents/Ketones/Xylene/Toluene/PCBTF | toys, asphalt coatings |
| | Block Copolymer | | and plastic |
| | (SEBS) | | modification |
| Palmer | 100% solids | Xylene/PCBTF/Mineral | Roof Coatings |
| Asphalt | asphalt | Spirits/Isoparafinnic Hydrocarbons | |
| Dow B66 | Thermoplastic | Xylene/PCBTF/Acetone | Clear aerosols, |
| | Acrylics | | concrete floors, |
| | | | gravure printing inks, |
| | | | ABS, polycarbonate, |
| | | | polystyrene coatings, |
| | | | maintenance |
| | | | coatings, specification |
| | | | lacquers |
| Pioneer | Thermoplastic | Xylene/PCBTF/Acetone | Oil and varnish |
| B67 | Acrylics | | modifier, General |
| | | | product finishing & |
| | | | topcote in metallizing |
| | | | applications |
| Pioneer MB | Thermoplastic | Xylene/PCBTF/Acetone | General purpose |
| 318 | Acrylics | | coating and ink |
| | | | applications |
| Neo Cryl | Thermoplastic | Xylene/PCBTF/Acetone | Clear aerosols, |
| 725 | Acrylics | | concrete floors, |
| | | | gravure printing inks, |
| | | | ABS, polycarbonate, |
| | | | polystyrene coatings, |
| | | | maintenance |
| | | | coatings, specification |
| | | | lacquers |
| Short Oil | | Xylene/Toluene/PCBTF | Industrial coatings |
| Alkyd | | | |
| Resyn | | | |
| Medium Ol | | Mineral | Architectural coatings |
| Alkyd | | Spirits/PCBTF/Xylene/VM&P/Toluene | |
| Resyn | | | |
| Long Oil | | Mineral | Architectural/Industrial |
| Alkyd | | Spirits/PCBTF/Xylene/VM&P/Toluene | coatings |
| Resyn | | | |
| Chain Stop | | Xylene/Toluene/Ketones/PCBTF | Industrial coatings |
| Alkyd & | | | |
| TOFA | | | |
| Curve | Lithium | Mineral Spirits | railcar, automotive, |
| Grease 213 | hydroxystearate | | aerospace, machinery |
| | grease | | lubricants |
| Motor Oil 30 | Petrochemical | Mineral Spirits | railcar, automotive, |
| Wt | derived | | aerospace, machinery |
| | | | engine lubricants |
| Synthetic | Derived from | Mineral Spirits | railcar, automotive, |
| Motor Oil | synthetic sources | | aerospace, machinery |
| | | | engine lubricants |
| Paraffin | Petrochemical & | Mineral Spirits/Methyl Ethyl | dewaxing light |
| Wax | synthetic Sources | Ketone/Xylene | lubricating oil stocks |
| | | | in pipelines, in |
| | | | formulation of |
| | | | candles, wax paper, |
| | | | polishes, cosmetics, |
| | | | and electrical |
| | | | insulators |

In the preceding description, for purposes of explanation, numerous details are set forth in order to provide a thorough understanding of the examples. However, it will be apparent to one skilled in the art that these specific details are not required.

The above-described examples are intended to be exemplary only. Alterations, modifications and variations can be effected to the particular examples by those of skill in the art without departing from the scope, which is defined by the claims appended hereto.

What is claimed is:

1. A method of reducing an amount of volatile organic components in a composition, the method comprising:
replacing at least a portion of a slow evaporating solvent in the composition with a compound of Formula (I): 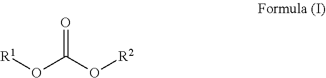
wherein R¹ and R² are independently: C₁₋₁₂ alkyl, C₂₋₁₂ alkenyl, C₂₋₁₂ alkynyl, C₆₋₁₄ aryl or siloxy, each of which are independently optionally substituted.

2. The method of claim 1, wherein the C₁₋₁₂ alkyl, C₂₋₁₂ alkenyl, C₂₋₁₂ alkynyl or C₆₋₁₄ aryl of the compound of Formula (I) are each independently substituted with a heteroatom.

3. The method of claim 2, wherein the heteroatom is N, O or S.

4. The method of claim 1, wherein R¹ or R² of the compound of Formula (I) are each independently hexyl, heptyl, octyl, or decyl.

5. The method of claim 1, wherein R¹ of the compound of Formula (I) is methyl and R² of the compound of Formula (I) is hexyl, heptyl, octyl, or decyl.

6. The method of claim 1, wherein R¹ of the compound of Formula (I) is methyl and R² of the compound of Formula (I) is 2-ethylhexyl.

7. The method of claim 1, wherein R¹ or R² of the compound of Formula (I) are each independently methyl or 2-ethylhexyl.

8. The method of claim 1, wherein R¹ or R² of the compound of Formula (I) are each independently 2-ethylhexyl.

9. The method of claim 1, wherein the slow evaporating solvent is an aliphatic solvent, an aromatic solvent, a glycol, or a ketone type solvent.

10. The method of claim 1, wherein the slow evaporating solvent is Mineral (white) Spirits, Aromatic 100, Aromatic 150, or Methyl Amyl Ketone.

11. The method of claim 1, wherein the compound of Formula (I) is a component or diluent in the production of a paint or a coating; a component in the formulation of general industrial primer, intermediate and/or topcoat; a component in the manufacture of a roof repair product; a component in the manufacture of a foundation repair product; a metal cleaner or a component thereof; a metal degreaser or a component thereof; a paint stripper or a component thereof; a diluent; a thickener; or a solvent.

12. The method of claim 11 wherein the paint or coating is an architectural paint or coating, a marine paint or coating, an aerospace paint or coating, an industrial paint or coating, a commercial paint or coating, a concrete paint or coating and/or a residential paint or coating.

13. The method of claim 11 wherein the solvent is a retarding solvent, an extraction solvent, a degreasing solvent, a cleaning solvent, or a component thereof.

14. The method of claim 13 wherein the cleaning solvent is an industrial cleaning solvent, a commercial cleaning solvent, or a residential cleaning solvent.

15. The method of claim 11 wherein the solvent is for dissolving tar or asphalt, for removing marks on flooring, or for removing carbon, grease, grime, gum, paint, adhesive or adhesive residue.

16. The method of claim 11 wherein the diluent or solvent is for use in liquid, aerosol paint, marking or adhesive formulations.

17. The method of claim 1, wherein the compound of Formula (I) is a sealant, a penetrant, a lubricant, a colourant diluent or a colourant dispersant.

18. The method of claim 1, wherein the compound of Formula (I) is a substitute for an ester alcohol, a reactive intermediate in the formation of an ester derivative, or an inert ingredient in the formulation of an insecticide, a fungicide and/or a rodenticide.

19. A method of forming a coating on a substrate, by incorporating a compound of Formula (I): 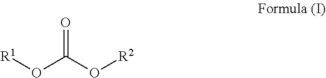
wherein R¹ and R² are independently: C₁₋₁₂ alkyl, C₂₋₁₂ alkenyl, C₂₋₁₂ alkynyl, C₆₋₁₄ aryl or siloxy, each of which are independently optionally substituted, into a paint.

20. A kit or commercial package comprising the compound of claim 1, together with instructions for use.

21. A method of preparing the compound of claim 1 as described in Example 2.
